(19) United States
(12) Patent Application Publication
(10) Pub. No.: US 2014/0147938 A1
(43) Pub. Date: May 29, 2014

(54) NON COVALENT MOLECULAR STRUCTURE, COMPRISING A PYRENE BASED GLYCOCONJUGATE, DEVICE COMPRISING THE SAME AND ITS USE FOR DETECTION OF LECTIN

(71) Applicants: Anne Imberty, Claix (FR); Sébastien Vidal, Villeurbanne (FR); Alxander Star, Pittsburgh, PA (US)

(72) Inventors: Anne Imberty, Claix (FR); Sébastien Vidal, Villeurbanne (FR); Alxander Star, Pittsburgh, PA (US)

(73) Assignees: CENTRE NATIONAL DE LA RECHERCHE SCIENTIFIQUE (CNRS), Paris cedex 16 (FR); University of Pittsburgh-Of the Commonwealth System of Higher Education, Pittsburgh, PA (US); INSA LYON, Villeurbanne Cedex (FR); UNIVERSITE CLAUDE BERNARD LYON 1 (UCBL), Villeurbanne Cedex (FR)

(21) Appl. No.: 14/131,330

(22) PCT Filed: Jul. 12, 2011
(86) PCT No.: PCT/IB2011/053100

§ 371 Date: Jan. 7, 2014

(51) Int. Cl. G01N 33/543; G01N 33/566; C07H 15/26

(52) U.S. Cl. CPC G01N 33/5438; C07H 15/26; G01N 33/566
USPC 436/501; 536/17.4; 422/69

(57) ABSTRACT

The present invention relates to a non covalent molecular structure comprising a carbon nanostructure and a pyrene based glycoconjugate (I) which is linked to the said carbon nanostructure by a non covalent link, the said glycoconjugate (I) having the formula: wherein B is a group which is present on any of the ten carbon atoms of the pyrene structure represented in (I) susceptible to bear a substituent, and is represented by the following group: —(CH2)ₙ-CO—NH-A, wherein n is an integer from 1 to 9, A is a group of formula: The present invention also relates to an electronic device comprising the said non covalent molecular structure, and to the use of this device for the detection of a lectin involved in bacterial or viral infections. Thus the invention also relates to a method for detecting the presence of a lectin in a sample to be analysed.

The present invention relates to novel non covalent molecular structures between carbon nanostructures and pyrene based glycoconjugates, to a device comprising these novel molecular structures and to the use of this device for the detection of a lectin.

Lectins are proteins capable of binding to carbohydrates but devoided of any catalytic activity and they are essential to many biological processes such as cell-to-cell communication, inflammation, viral infections (HIV, influenza), cancer or bacterial adhesion. Lectins are specialized receptors which are used by several opportunistic Gram negative bacteria for specific recognition of human glycans present on tissue surface. Most lectins from opportunistic bacteria bind complex oligosaccharides such as the ones defining histo-blood group epitopes. Contrary to their counterpart in plants or animals, bacterial lectins present strong affinity towards ligands which makes them attractive targets for diagnostic.

The detection of bacterial lectins is required in the case of bacterial or viral infections and is of primary importance for public health but is also of importance in hospitals for safety purposes (most of hospital acquired infections being caused by bacteria with about 20% of these due to Pseudomonas aeruginosa) and the prevention of exposure to these agents. This is also true for outdoor environmental safety issues like the prevention of exposure to these agents through recreative waters (public swimming pools, lakes, others water reservoirs), tap waters and even for the prevention of biological terrorism.

At the present time, the detection of bacteria is classically achieved through culture-based techniques or through molecular techniques based on polymerase chain reaction (PCR). However both methods are relatively slow and not always applicable (non-culturable bacteria, impurity in DNA samples . . . ). These molecular methods can take up to a few days and require specialized skills.

An alternative to these techniques can be the use of nano-technologies for designing miniaturized and highly sensitive bioanalytical systems. The fast growing field of nanotechnology has found several applications in cell biology through quantum dots, nanofibers and carbon nanotubes.

Single-walled carbon nanotubes (SWNTs) are ideal for the design of biosensors because of their high electrical conductivity and small diameter (˜1 nm) which is comparable to the size of individual biomolecules. Additionally, SWNTs are composed almost entirely of surface atoms allowing detection of tiny changes in their local chemical environment and thus display extreme sensitivity. These unique attributes have led researchers to incorporate SWNTs as conductive channels in solid-state electronic devices such as field-effect transistors (FETs), creating low power and ultra small electro-analytical platforms for monitoring various biomolecular interactions.

The WO 2008/044896 document relates to carbon nanotubes (CNT)-Dendron composite and a biosensor for detecting a biomolecule comprising the CNT-Dendron composite.

The WO 2009/141486 document relates to a glycolipid/carbon nanotube aggregate and to the use thereof in processes that involve interactions between carbohydrates and other biochemical species.

However none of these documents relate to the detection of lectins.

The publication “Assali M and al., Royal Society of Chemistry, Vol. 5, no. 5, 2009, p. 948-950”, describes the utilization of neutral pyrene functionalized neoglycolipids that interact with a carbon nanotube surface giving rise to biocompatible nanomaterials which are able to engage specific ligand-lectin interactions similar to glycoconjugates on the cell membrane. The authors of this document addressed the question of binding between the functionalized nanotubes and lectins by using fluorescence spectroscopy.

However nothing is said in this document about a detection of lectins which would be based on the specific conductance of carbon nanotubes, and which would be fast, accurate, quantitative and which has an excellent sensitivity.

Therefore, there is a need to develop advantageous diagnostic methods permitting the detection of lectins.

One aim of the invention is to provide a method for detecting the presence of a lectin involved in bacterial or viral infections which is fast (less than 1 minute), accurate and quantitative.

Another aim of the invention is to provide a novel diagnostic method of a bacterial lectin having an excellent sensitivity.

Another aim of the invention is to provide an accurate and rapid diagnostic of the presence or not of a lectin from all bacteria, viruses and parasites that use human glycoconjugates in the early steps of infection.

In an aspect, the present invention provides a non covalent molecular structure characterized in that it comprises a carbon nanostructure and a pyrene based glycoconjugate (I) which is linked to the said carbon nanostructure by a non covalent link, the said glycoconjugate (I) having the formula:

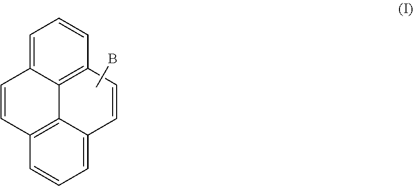

wherein
B is a group which is present on any of the ten carbon atoms of the pyrene structure represented in (I) susceptible to bear a substituent, and is represented by the following group:

—(CH₂)ₙ—CO—NH-A,

wherein

n is an integer from 1 to 9,

A is a group of formula:

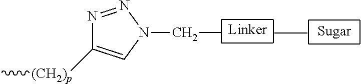

wherein
p is an integer from 1 to 9,
the [symbol] is a group of formula:

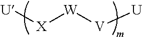

wherein
m is an integer from 0 to 15,
U′, U=absent or is CH₂ with the proviso that when m=0 then if one of U′ or U is absent then the other is CH₂,
X=CH₂, O, CO (carbonyl),
W=CH₂, NH,
V=CH₂, C₆H₄ (phenyl “Ph”),
the [symbol] is a group having at least one carbohydrate moiety and is selecting in the group comprising:

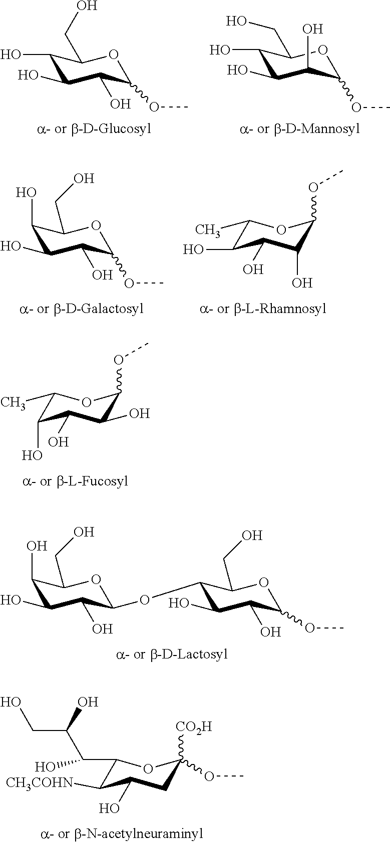

and their derivatives.
   The pyrene based glycoconjugate (I) according to the present invention can also be represented by the following formula:

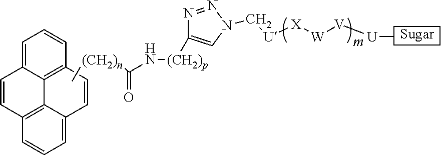

Advantageously, the above mentioned sugar derivatives defined in the A group are for example selected in the group comprising:

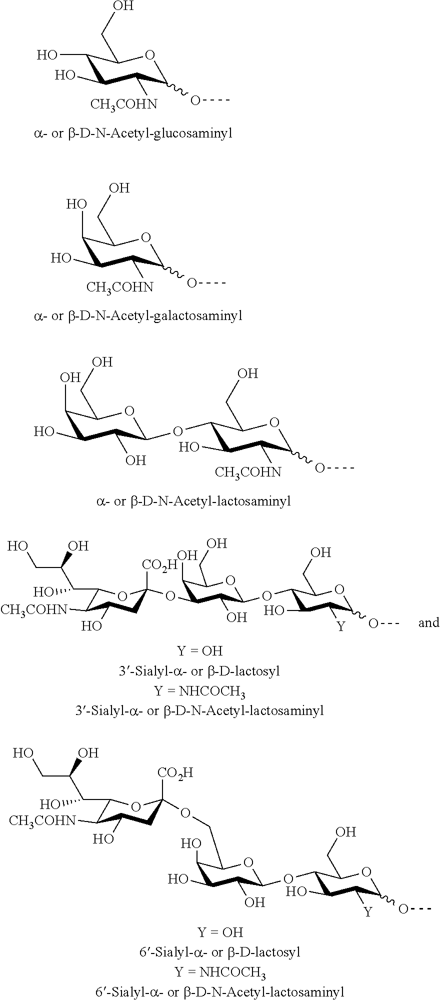

In another aspect, the above mentioned sugar derivatives defined in the A group are selected in the group comprising:

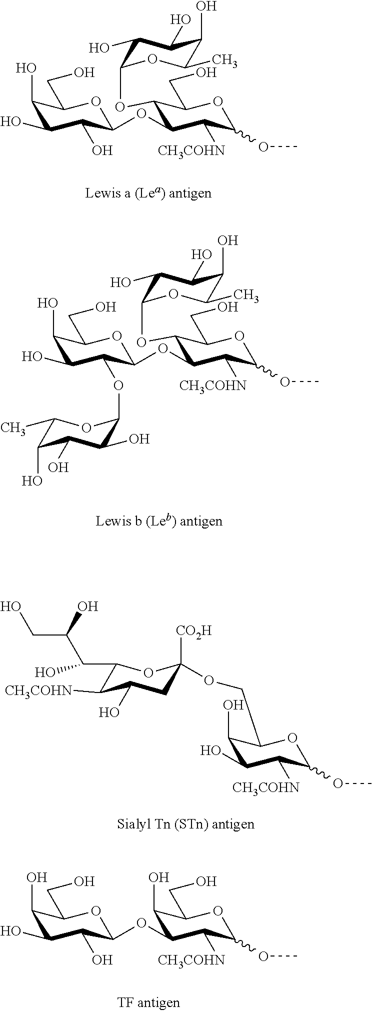

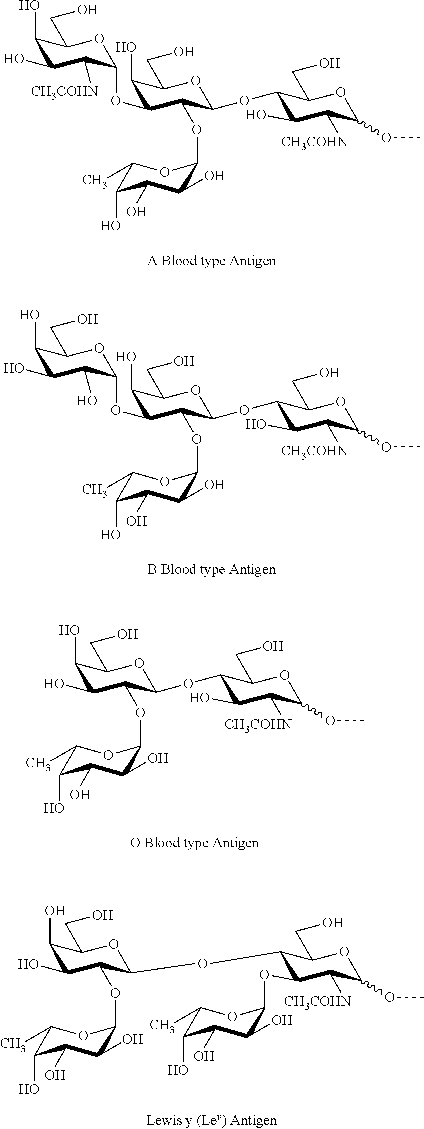

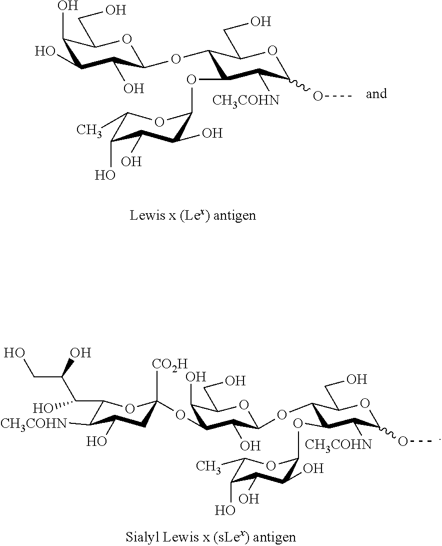

The wave bond situated between the anomeric carbon atom and the exocyclic oxygen atom means that the stereochemistry can be either alpha or beta (axial or equatorial)
Advantageously, the [symbol] defined in the A group of the non covalent molecular structure is selected in the group comprising

m=0, U′=absent and U=CH₂(i.e. [symbol] =CH₂),

m=0, U′=U=CH₂(i.e. [symbol] =(CH₂(₂)

m=1, U′=U=absent, X=W=V=CH₂(i.e. [symbol] =(CH₂) ₃),

m=2, U′=U=absent, X=W=V=CH₂(i.e. [symbol] =(CH₂)₆),

m=1, U′=CH₂, U=absent, X=O, W=V=CH₂(i.e. [symbol] =CH₂—(O—CH₂—CH₂—CH₂)),

m=2, U′=CH₂, U=absent, X=O, W=V=CH₂(i.e. [symbol] =CH₂—(O—CH₂—CH₂—CH₂)₂),

m=2, U′=absent, U=V=CH₂, X=CO, W=NH (i.e. [symbol] =(CO—NH—CH₂)₂—CH₂) and

m=1, U′=U=absent, X=CO, W=NH and V=Ph (i.e. [symbol] =CO—NH—Ph).

In a further aspect of the invention, in the pyrene based glycoconjugate (I) of the non covalent molecular structure, the integer n is 3, the integer p is 1 and the said glycoconjugate (I) is represented by the formula:

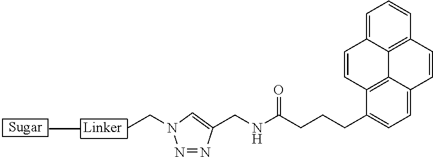

In yet a further aspect of the invention, in the pyrene based glycoconjugate (I) of the non covalent molecular structure as defined above, the [symbol] is CH₂—(O—CH₂—CH₂)₂ and the sugar is selected in the group comprising β-D-galactosyl, α-D-mannosyl and α-L-fucosyl.

In another aspect of the present invention, the carbon nanostructures of the non covalent molecular structure are selected in the group comprising carbon nanotubes, graphene, graphitic onions, cones, nanohorns, nanohelices, nanobarrels and fullerenes.

Advantageously, the above mentioned carbon nanostructures are preferably graphene or carbon nanotubes, the said carbon nanotubes being selected in the group comprising Single Wall Carbon Nanotubes (SWCNTs), Double Wall Carbon Nanotubes (DWCNTs), Triple Wall Carbon Nanotubes (TWCNTs) and Multi Wall Carbon Nanotubes (MWCNTs).

Graphene is a one-atom-thick planar sheet of sp²-bonded carbon atoms that are densely packed in a honeycomb crystal lattice.

The present invention also provides any device comprising a non covalent molecular structure as defined previously and capable of detecting a lectin in an aqueous solution through an electrical resistivity or conductivity.

Thus in another aspect, the present invention provides a device for detecting a lectin characterized in that it comprises a non covalent molecular structure as defined previously.

According to an aspect of the present invention, such a device could advantageously be an electronic nano-detection device comprising a field effect transistor (FET), the said device comprising:

carbon nanostructures bridging two metal electrodes respectively called “source” (S) and “drain” (D),

a third electrode called “gate” (G) connected either to a substrate layer or to an electrode immersed in a solution covering the said device (“liquid gate”).

One of the originality of the present invention is thus the use of the said non covalent molecular structure in a device as above described for the detection of a lectin involved in bacterial or viral infections. The Inventors of the present invention have advantageously combined several knowledges of different technical fields in order to establish novel molecular structures which can be used for a diagnostic purpose (the detection of a bacterial lectin).

Thus here is used—biological knowledges about the capacity of some pathogens (bacterial lectins) to attach to human glycans (glycolipids and glycoproteins) present at the surface of human cells (that is to say the carbohydrate-lectin interactions involved in bacterial virulence)—knowledges concerning nanotechnology and the electronic devices and chemical knowledges in order to conceive a chemical structure which will interact with the electronic device and the lectins.

The originality of the invention consists thus to use glycoconjugate structures linked to carbon nanostructures in a field effect transistor (FET) device in order to provide a device for detecting a lectin which is very advantageous.

In the device as described previously, the two metal electrodes (S) and (D) are spacing each other from 1 nm to 10 cm, preferably from 1 cm to 2.5 cm and more preferably from 1 μm to 10 μm.

Any metal is appropriate for preparing the electrodes (S) and (D). Examples of suitable metal can include, but are not limited to aluminium, chromium, titanium, gold and palladium.

Advantageously in the said device, the substrate layer is an insulator. Examples of suitable substrate layers can include, but are not limited to silicon dioxide layer, hafnium oxide and silicon nitrate.

According to still another aspect, the present invention also provides a method for detecting the presence of a lectin in a sample to be analysed characterized in that it comprises the following steps:

using a device as described previously,

bringing the lectin to be analysed in contact with the non covalent molecular structure as described previously,

detecting a molecular interaction between the lectin and the sugar of the pyrene based glycoconjugate (I) of the said non covalent molecular structure, said molecular interaction being detected by a change of the conductive properties of the carbon nanostructures resulting in a change of the electric signal of the said device.

Advantageously, according to the present invention, the pyrene based glycoconjugates (I) will be used for selective attachment of targeted lectins while carbon nanostructures with their nanoscale dimensions, large surface to volume ratio and unique physical and chemical properties will aid in electronic transduction of the interaction between glycoconjugates and lectins, leading to a rapid and ultrasensitive detection.

The change in carbon nanostructures-FET conductance will be used for studying the molecular interaction between pyrene based glycoconjugate (I) and lectin as well as to monitor the variation in lectin concentration.

The sample to be analysed can come from a pure lectin from commercial sources or isolated from recombinant production techniques, or any sample containing bacteria such as water, soils or sample of human origin.

In a general way, the method according to the present invention can be used for the detection of lectins from all bacteria, viruses and parasites that use human glycoconjugates in the early steps of infection. Advantageously, examples of suitable lectins can include, but are not limited to, those selected in the group comprising Pseudomonas aeruginosa first lectin (PA-IL), Pseudomonas aeruginosa second lectin (PA-IIL), Concanavalin A (Con A) lectin, Burkholderia cenocepacia A (Bc2L-A) lectin, Burkholderia cenocepacia B (Bc2L-B) lectin, Burkholderia cenocepacia C (Bc2L-C) lectin, Burkholderia ambifaria (Bamb541) lectin, Ralstonia solanacearum (RSL) lectin, Ralstonia solanacearum second lectin (RS-IIL) and Chromobacterium violaceum (CV-IIL) lectin.

In another aspect of the invention, the preparation of the device as above defined comprises the following steps:

forming two metal electrodes (S) and (D) on the substrate layer connected to (G),

adding, between the two electrodes (S) and (D), the carbon nanostructures and then a pyrene based glycoconjugate (I) in order to form a non covalent molecular structure as defined.

In a further aspect of the invention, the preparation of the device as above defined comprises the following steps:

forming two metal electrodes (S) and (D) on the substrate layer connected to (G),

adding, between the two electrodes (S) and (D), a non covalent molecular structure as above defined.

In yet a further aspect of the invention, the preparation of the device as above defined comprises the following steps:

generating carbon nanostructures on the substrate layer connected to (G) (by a chemical vapour deposition (CVD) process),

forming two metal electrodes (S) and (D) around the carbon nanostructures,

adding a pyrene based glycoconjugate (I) in order to form a non covalent molecular structure as above defined.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention.

Reference is now made to the following examples in conjunction with the accompanying drawings.

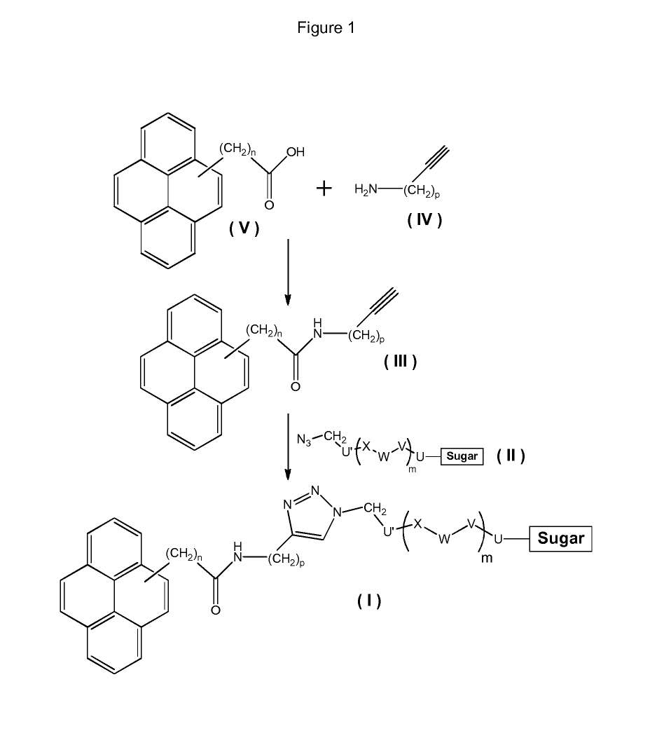

FIG. 1 is a general synthesis scheme illustrating the chemical structures and the preparation of pyrene based glycoconjugates (I).

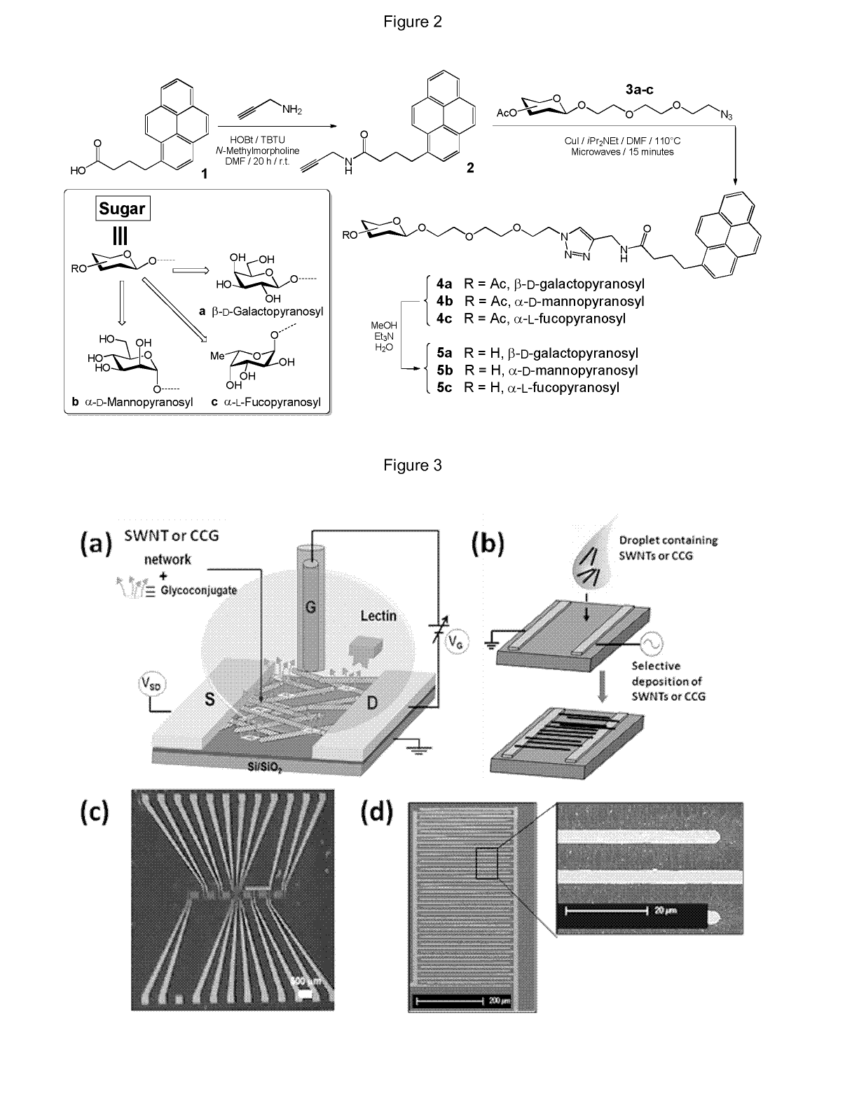

FIG. 2 represents a specific synthesis scheme (illustrating the general synthesis scheme of FIG. 1) of three pyrene based glycoconjugates (I) wherein:

n=3,

[symbol] =CH₂—(O—CH₂—CH₂)₂,

[symbol] =β-D-galactosyl (see compound named 5a) or α-D-mannosyl (compound 5b) or α-L-fucosyl (compound 5c).

“Ac” (which is defined in compounds 4a to 4c) representing the “acetyl” radical (CO—CH₃).

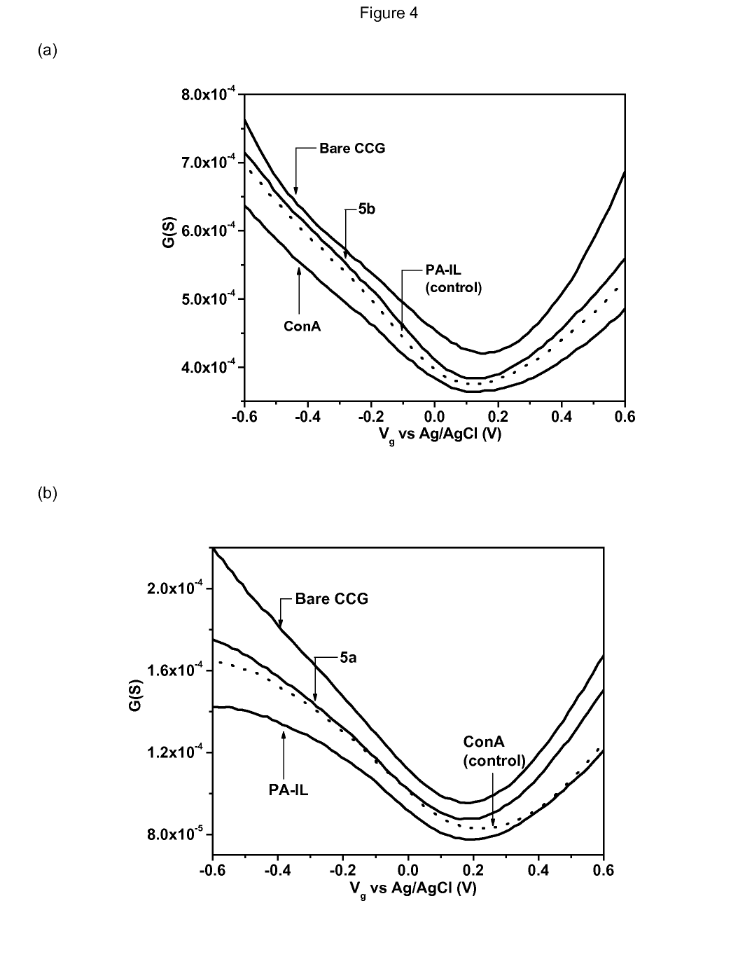

FIG. 3 represents a “SWNT-FET” device (SWNT=“single wall carbon nanotubes” and FET=“Field Effect Transistor”) or a “CCG-FET” device (CCG=chemically converted graphene) and its fabrication. More particularly FIG. 3(a) is a schematic illustration of glycoconjugate (I) functionalized single walled carbon nanotubes (SWNTs)-FET detection platform or of glycoconjugate (I) functionalized chemically converted graphene (CCGs)-FET detection platform for selective detection of lectin. FIG. 3(b) is a schematic of dielectrophorectic method used for selective deposition of SWNTs or of CCGs onto pre-patterned microelectrodes. FIG. 3(c) is an optical image of Si/SiO₂ chip with micropatterned interdigitated electrodes. FIG. 3(d) is a SEM image of interdigitated electrodes used for device fabrication. Inset shows the SWNTs or the CCGs deposited by dielectrophoresis technique between microelectrodes.

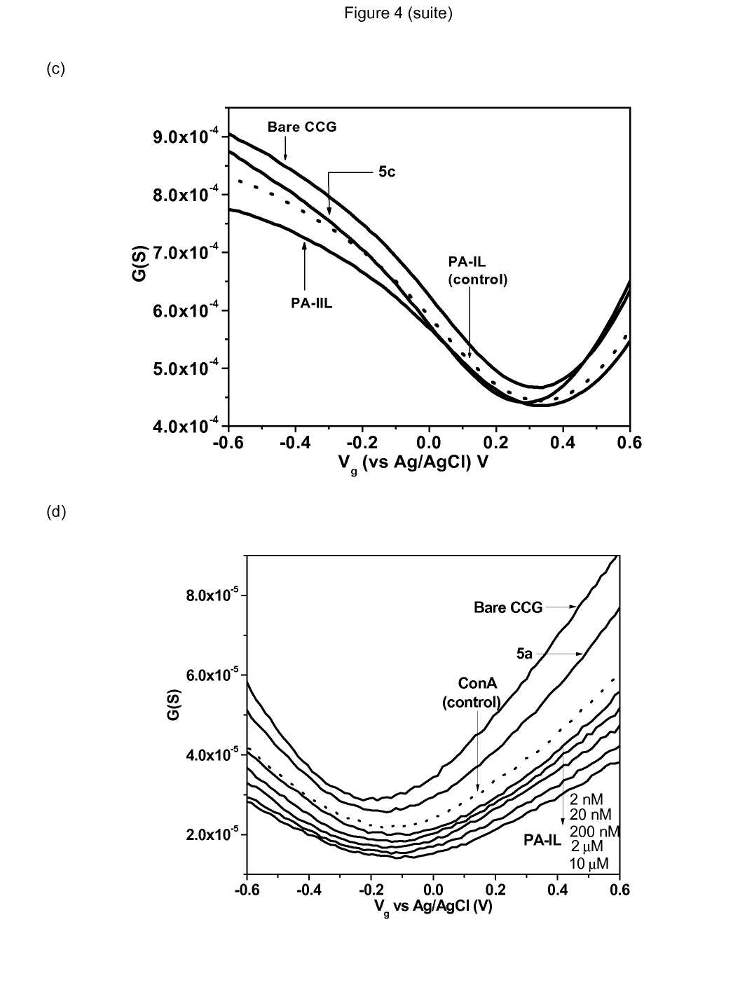

FIG. 4 represents the electronic detection of carbohydrate-lectin interactions. More particularly, FIG. 4 shows the conductance “G” (which is expressed in siemens (S)) versus gate voltage (“Vg”) of bare CCG-FET device and after functionalization with respectively the α-D-mannose pyrene based glycoconjugate 5b (FIG. 4(a)), the R-D-galactose pyrene based glycoconjugate 5a (see FIG. 4(b)) and the α-L-fucose pyrene based glycoconjugate 5c (see FIG. 4(c)) and after incubation with 2 μM non-selective lectin (control) and 2 μM selective lectin. PA-IL will be a lectin selective for R-D-galactose and non-selective for α-D-mannose or α-L-fucose. Con A will be a lectin selective for α-D-mannose and non-selective for R-D-galactose. PA-IIL will be a lectin selective for α-L-fucose.

FIG. 4(d) represents the same experiment as in FIG. 4(b) but with 10 μM ConA as the control and varying concentration of the selective lectin (PA-IL) (2 nM-10 μM).

All measurements were performed in electrolyte-gated FET configuration in PBS (pH 7), Ag/AgCl reference electrode, with source-drain voltage of 50 mV.

Lectin binding experiments were performed in the presence of 5 μM Ca²⁺.

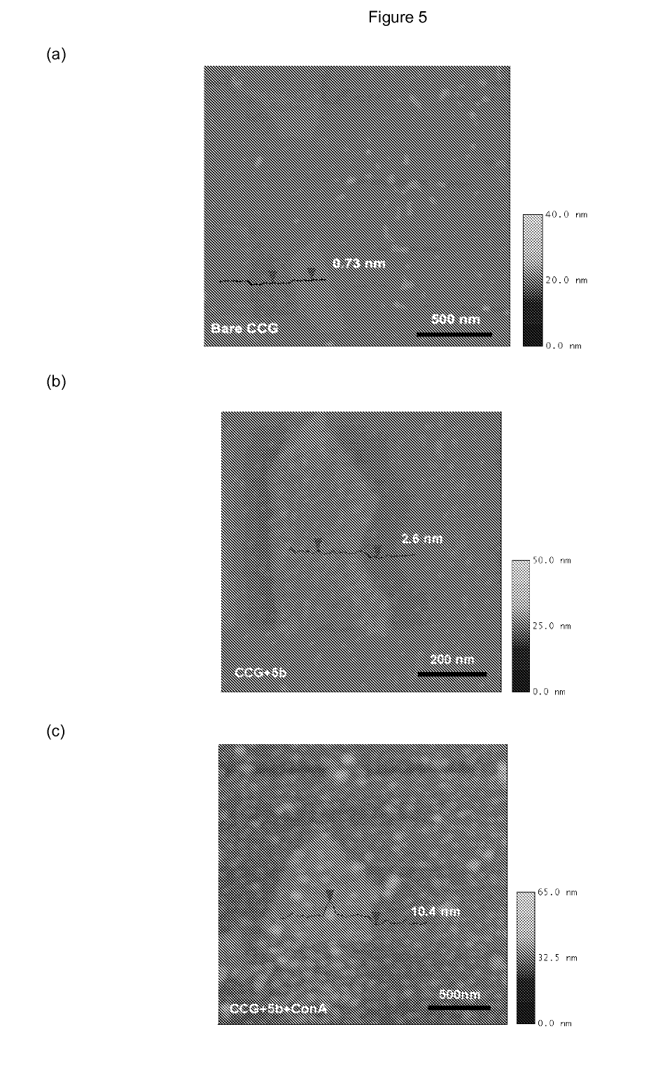

FIG. 5 shows Atomic Force Microscope (AFM) images from bare CCG (FIG. 5(a)), from CCG functionalized with α-D-mannose pyrene based glycoconjugate 5b (defined as “COG-51D”) (FIG. 5(b)) and after ConA lectin attachment (defined as “CCG-5b-ConA”) (FIG. 5(c)). Lectin attachment was performed in the presence of 5 μM Ca²⁺.

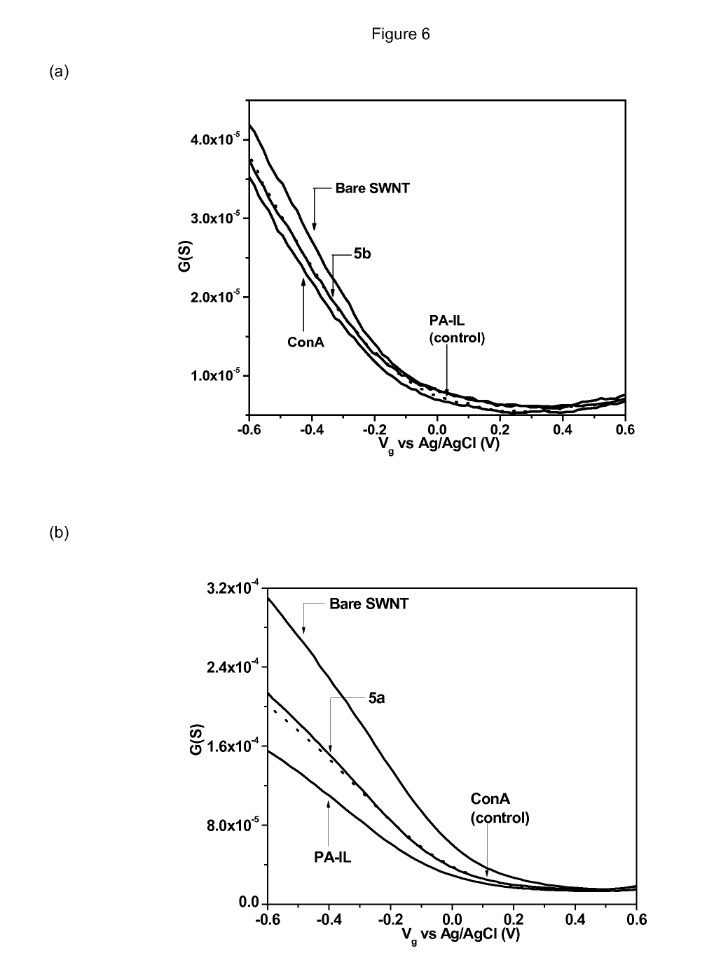

FIG. 6 represents the electronic detection of carbohydrate-lectin interactions. More particularly, FIG. 6 shows the conductance “G” (which is expressed in siemens (S)) versus gate voltage (“Vg”) of bare SWNT-FET device and after functionalization with respectively the α-D-mannose pyrene based glycoconjugate 5b (FIG. 6(a)) and the R-D-galactose pyrene based glycoconjugate 5a (FIG. 6(b)) and after attachment with 2 μM non-selective lectin (control) and 2 μM selective lectin.

Lectin attachment was performed in the presence of 5 μM Ca²⁺.

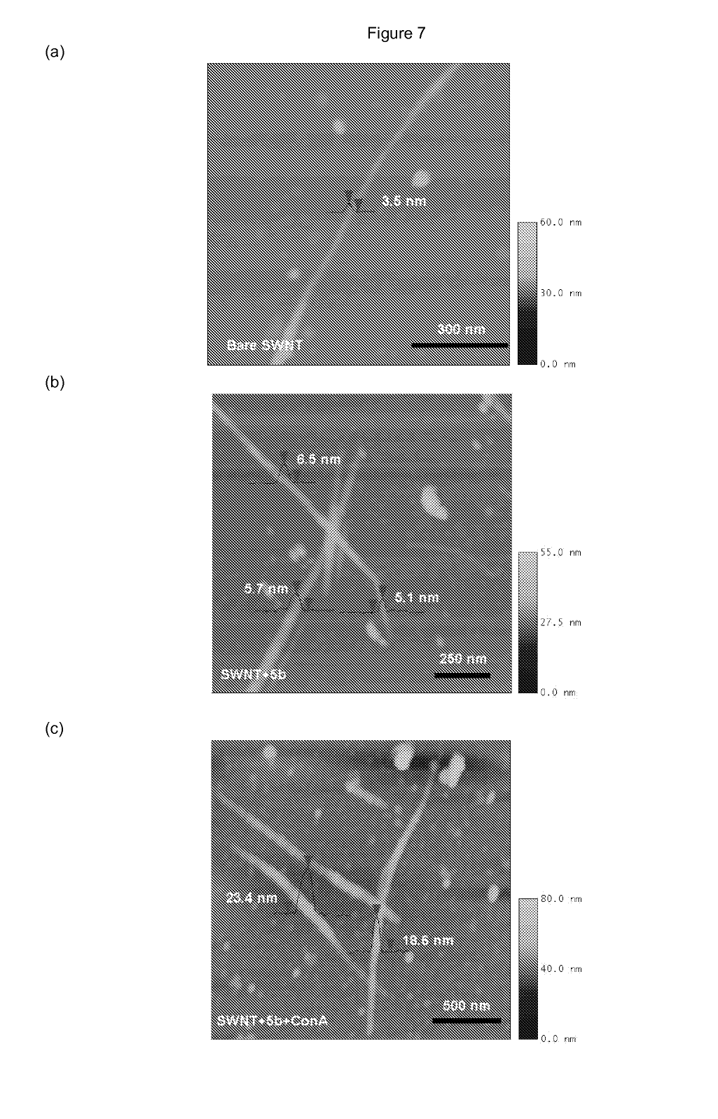

FIG. 7 shows Atomic Force Microscope (AFM) images from bare SWNTs (FIG. 7(a)), from SWNT functionalized with the α-D-mannose pyrene based glycoconjugate 5b (defined as “SWNT-5b”) (FIG. 7(b)) and after ConA lectin attachment (defined as “SWNT-5b-ConA”) (FIG. 7(c)). Lectin attachment was performed in the presence of 5 μM Ca²⁺.

### EXAMPLE I

### Preparation of Three Pyrene Glycoconjugates (I)

The general synthesis scheme used in this example for preparing the pyrene based glycoconjugates of general formula (I) is illustrated in FIG. 1, wherein an alkynyl-amine of general formula (IV) is condensed with a pyrene-based carboxylic acid of general formula (V) leading to an alkynyl amide of general formula (III) which is then conjugated with a carbohydrate azido-derivative of general formula (II) to afford the pyrene based glycoconjugate of general formula (I).

General experimental methods are described for preparing the three following pyrene based glycoconjugate (I):
N-[1-(2-{2-[2-(β-D-Galactopyranosyloxyethoxy)ethoxy]ethyl}-1H-1,2,3-triazol-4-yl)methyl]-4-(pyren-1-yl)butanamide (named 5a in FIG. 2),
N-[1-(2-{2-[2-(β-D-Mannopyranosyloxyethoxy)ethoxy]ethyl}-1H-1,2,3-triazol-4-yl)methyl]-4-(pyren-1-yl)butanamide (named 5b in FIG. 2) and,
N-[1-(2-{2-[2-(α-L-Fucopyranosyloxyethoxy)ethoxy]ethyl}-1H-1,2,3-triazol-4-yl)methyl]-4-(pyren-1-yl)butanamide (named 5c in FIG. 2).

All reagents were commercial (highest purity available for reagent grade compounds) and used without further purification. Solvents were distilled over CaH₂ (CH₂Cl₂) or Mg/I₂ (MeOH).

Reactions were performed under an argon atmosphere. Reactions under microwave activation were performed on a Biotage Initiator system.

Thin-layer chromatography (TLC) was carried out on aluminum sheets coated with silica gel 60 F₂₅₄ (Merck). TLC plates were inspected by UV light (λ=254 nm) and developed by treatment with a mixture of 10% H₂SO₄ in EtOH/H₂O (95:5 v/v) followed by heating.

Silica gel column chromatography was performed with silica gel Si 60 (40-63 μm).

NMR spectra were recorded at 293 K, unless otherwise stated, using a 300 MHz or a 400 MHz Bruker Spectrometer. Chemical shifts are referenced relative to deuterated solvent residual peaks. The following abbreviations are used to explain the observed multiplicities: s, singlet; d, doublet; t, triplet; q, quadruplet; m, multiplet and bs, broad singlet.

A residual peak at 147.8 ppm was due to the machine and could be usually observed on 75 MHz ¹³C spectra. This residual peak was checked to be independent from the sample analyses. Complete signal assignments were based on 1D and 2D NMR experiments (COSY, HSQC and HMBC). High-resolution (HR-ESI-QTOF) mass spectra were recorded by using a Bruker MicrOTOF-Q II XL spectrometer. The carbohydrate azido-derivatives named 3a,¹ 3b,² and 3c³ were previously described in the literature and prepared accordingly.

1) General Procedure for 1,3-Dipolar Cycloadditions (Method A)

The alkyne-functionalized pyrene derivative 2 (of general formula (III)), copper iodide, N,N-diisopropylethylamine (DIPEA) and azido-derivatives 3a to 3c (of general formula (II)) in degassed DMF were introduced in a Biotage Initiator 2-5 mL vial. The vial was flushed with argon and protected from light (aluminum sheet) and the solution was sonicated for 30 seconds. The vial was sealed with a septum cap and heated at 110° C. for 10 min under microwave irradiation (solvent absorption level: high). After uncapping the vial, the crude mixture was evaporated then purified by flash silica gel column chromatography to afford the desired acetylated pyrene glycoconjugate 4a to 4c.

2) General Procedure for Deacetylation (Method B)

The acetylated pyrene glycoconjugate 4a to 4c were suspended in distilled MeOH, ultra-pure water and ultra-pure triethylamine (10:1:1, v/v/v). The mixture was stirred under argon at room temperature for 1 to 3 days. Solvents were evaporated off then co-evaporated with toluene. The residue was dissolved in ultra-pure water (5 mL) and freeze-dried to afford pure hydroxylated pyrene glycoconjugates 5a to 5c (general formula (I)).

The synthesis scheme of the three pyrene glycoconjugates 5a to 5c is illustrated in FIG. 2. The reagents and conditions used in the steps described in FIG. 2 are given below:
Step a: N-hydroxy-benzotriazole (HOBt)/O-(Benzotriazol-1-yl)-N,N,N′,N′-tetramethyluronium tetrafluoroborate (TBTU), N-methylmorpholine, N,N-dimethylformamide (DMF)/20 h/r.t.;
Step b: copper iodide (CuI), N,N-diisopropylethylamine, DMF, 110° C., Microwaves, 15 minutes;
Step c (deacetylation): MeOH, triethylamine (Et₃N), H₂O.

### (a) Preparation of Compound 2 (General Formula (III))

### N-(Propargyl)-4-(Pyren-1-Yl)Butanamide

N-Methylmorpholine (3.8 mL, 34.7 mmol) was added to a solution of 1-pyrenebutyric acid 1 (2 g, 6.9 mmol), TBTU (8.9 g, 27.7 mmol), and HOBt (3.75 g, 27.7 mmol) in DMF (80 mL). The solution was stirred at RT for 15 min then propargyl amine (2.22 mL, 34.7 mmol) was added and the reaction stirred at RT for an additional 16 h. The solution was poured into EtOAc (700 mL) then washed with saturated NaHCO₃ (2×200 mL) and water (200 mL). The organic layer was dried (MgSO₄), filtered and evaporated. The crude mixture was purified by silica gel column chromatography (CH₂Cl₂/EtOAc 2/1). The product 2 was obtained pure (1.52 g, 67%) after precipitation from CH₂Cl₂/Petroleum ether.

R_{f}=0.71 (CH₂Cl₂/EtOAc 2/1)

M.p.=147-149° C.

The ¹H NMR and ¹³C NMR data are given below.

¹H NMR (400 MHz, DMSO-d₆):

δ 8.37 (d, J=9.3 Hz, 1H, H-ar), 8.33 (t, J=5.3 Hz, 1H, NH), 8.29-8.17 (m, 4H, H-ar), 8.11 (d, J=1.8 Hz, 1H, H-ar), 8.04 (t, J=7.7 Hz, 1H, H-ar), 7.92 (d, J=7.7 Hz, 1H, H-ar), 3.90 (dd, 2H, J=2.4 Hz, J=5.4 Hz, NCH₂), 3.31 (t, 2H, J=7.4 Hz, PyrCH₂CH₂CH₂C(O)), 3.12 (t, 1H, J=2.4 Hz, C≡CH), 2.27 (t, 2H, J=7.4 Hz, PyrCH₂CH₂CH₂C(O)), 2.05-1.98 (m, 2H, PyrCH₂CH₂CH₂C(O)).

¹³C NMR (100 MHz, DMSO-d₆):

δ 171.7 (C═O), 136.5, 130.9, 130.4, 129.3, 128.1 (C^{IV}-ar), 127.5, 127.4, 127.2, 126.5, 126.1, 124.9, 124.8 (CH-ar), 124.2, 124.1 (C^{IV}-ar), 123.5 (CH-ar), 81.4 (C≡CH), 72.8 (C≡CH), 34.7 (PyrCH₂CH₂CH₂C(O)), 32.2 (PyrCH₂CH₂CH₂C(O)), 27.8 (NCH₂), 27.4 (PyrCH₂CH₂CH₂C(O)).

### (b) Preparation of Compound 4a (General Formula (I°)

### N-[1-(2-{2-[2-(2,3,4,6-Tetra-O-acetyl-β-D-galactopyranosyloxyethoxy)ethoxy]ethyl}-1H-1,2,3-triazol-4-yl)methyl]-4-(pyren-1-yl)butanamide

This compound is prepared according to method A in 47% yield.

R_{f}=0.25 (EtOAc/MeOH 95/5)

The ¹H NMR and ¹³C NMR data are given below.

¹H NMR (400 MHz, CDCl₃:

δ 8.25 (d, 1H, J=8.8 Hz, H-ar), 8.16-8.12 (m, 2H, H-ar), 8.07 (d, 2H, J=7.6 Hz, H-ar), 8.00 (s, 1H, H-triaz), 7.97 (t, 3H, J=7.6 Hz, H-ar), 7.82 (d, 1H, J=7.6 Hz, H-ar,), 6.60-6.40 (bs, 1H, NH), 5.36 (d, 1H, J=3.6 Hz, H-4), 5.16 (dd, 1H, J=7.8 Hz, J=10.4 Hz, H-2), 5.00 (dd, 1H, J=3.6 Hz, J=10.4 Hz, H-3), 4.60-4.48 (m, 4H, OCH₂CH₂N-triaz, CCH₂NH), 4.47 (d, 1H, J=7.8 Hz, H-1), 4.16-4.04 (m, 2H, H-6), 3.91-3.76 (m, 4H, H-5, ½ GalOCH₂CH₂O, OCH₂CH₂N-triaz), 3.64-3.60 (m, 1H, ½ GalOCH₂CH₂O), 3.53-3.42 (m, 6H, GalOCH₂CH₂OCH₂CH₂O), 3.35, 2.36, 2.20 (3bs, 6H, PyrCH₂CH₂CH₂C(O)), 2.11, 2.00, 1.99, 1.96 (4s, 4×3H, CH₃CO).

¹³C NMR 100 MHz, CDCl₃):

δ 170.5, 170.3, 170.2, 169.6, (4s, 4C, C═O), 135.9, 131.5, 131.0, 130.0, 128.8 (C^{IV}-ar), 127.6 (CH-ar), 127.47 (s, 2C, CH-ar, CH-triaz), 127.46 (CH-ar), 126.0, 125.9 (CH-ar), 125.1, 125.03 (C^{IV}-ar), 124.99, 124.89, 124.86, 123.5 (CH-ar), 101.4 (C-1), 70.9 (C-3), 70.7 (C-5), 70.62, 70.58, 70.2 (3s, 3C, GalOCH₂CH₂OCH₂CH₂O), 69.3 (OCH₂CH₂N-triaz), 69.2 (GalOCH₂CH₂O), 68.9 (C-2), 67.3 (C-4), 61.3 (C-6), 50.9 (OCH₂CH₂N-triaz), 32.9, 27.5 (PyrCH₂CH₂CH₂C(O)), 20.9, 20.8, 20.7 (3s, 4C, CH₃CO).

### (c) Preparation of Compound 4b (General Formula (I))

### N-[1-(2-{2-[2-(2,3,4,6-Tetra-O-acetyl-β-D-mannopyranosyloxyethoxy)ethoxy]ethyl}-1H-1,2,3-triazol-4-yl)methyl]-4-(pyren-1-yl)butanamide

This compound is prepared according to method A in 99% yield.

R_{f}=0.23 (EtOAc/MeOH 95/5)

The ¹H NMR and ¹³C NMR data are given below.

¹H NMR (400 MHz, CDCl₃):

δ 8.23 (d, J=9.2 Hz, 1H, H-ar), 8.13 (d, J=1.6 Hz, 1H, H-ar), 8.11 (d, J=1.6 Hz, 1H, H-ar), 8.05 (d, J=8.2 Hz, 2H, H-ar), 7.98 (s, 1H, H-triaz), 7.95 (t, J=7.7 Hz, 3H, H-ar), 7.79 (d, J=7.7 Hz, 1H, H-ar), 6.66 (bs, 1H, NH), 5.33-5.25 (m, 2H, H-3, H-4), 5.24-5.21 (m, 1H, H-2), 4.82 (d, J=1.3 Hz, 1H, H-1), 4.52 (bs, 2H, CCH₂NH), 4.45 (bs, 2H, OCH₂CH₂N-triaz), 4.25 (dd, J=12.3 Hz, J=5.0 Hz, 1H, H-6b), 4.14-4.05 (m, 1H, H-6a), 4.04-3.97 (m, 1H, H-5), 3.78 (bs, 2H, OCH₂CH₂N-triaz), 3.74-3.66 (m, 1H, ½ ManOCH₂CH₂O), 3.60-3.52 (m, 1H, ½ ManOCH₂CH₂O), 3.52-3.44 (m, 6H, ManOCH₂CH₂OCH₂CH₂O), 3.32 (t, J=7.0 Hz, 2H, PyrCH₂CH₂CH₂C(O)), 2.32, 2.17 (2 bs, 4H, PyrCH₂CH₂CH₂C(O)), 2.12, 2.07, 2.01, 1.96 (4s, 4×3H, CH₃CO).

¹³C NMR (100 MHz, CDCl₃):

δ 170.7, 170.14, 170.07, 169.8 (4s, 4C, CH₃CO), 135.9 (C^{IV}-ar), 131.4 (C^{IV}-ar), 130.9 (C^{IV}-ar), 129.9 (C^{IV}-ar), 128.8 (C^{IV}-ar), 127.5 (CH-ar), 127.40 (s, 2C, CH-triaz, CH-ar), 127.41 (CH-ar), 126.7 (CH-ar), 125.9 (CH-ar), 125.1 (C^{IV}-ar), 125.0 (C^{IV}-ar), 124.9 (CH-ar), 124.85 (CH-ar), 124.81 (CH-ar), 123.4 (CH-ar), 97.7 (C-1), 70.6, 70.5, 69.9 (3s, 3C, ManOCH₂CH₂OCH₂CH₂O), 69.6 (C-2), 69.4 (OCH₂CH₂N-triaz), 69.1 (C-3), 68.5 (C-5), 67.3 (ManOCH₂CH₂O), 66.1 (C-4), 62.5 (C-6), 50.5 (OCH₂CH₂N-triaz), 36.1 (PyrCH₂CH₂CH₂C(O)), 34.9 (CCH₂NH), 32.8 (PyrCH₂CH₂CH₂C(O)), 27.5 (PyrCH₂CH₂CH₂C(O)), 21.0, 20.82, 20.77 (3s, 4C, CH₃CO).

### (d) Preparation of Compound 4c (General Formula (I))

### N-[1-(2-{2-[2-(2,3,4-Tri-O-acetyl-α-L-fucopyranosyloxyethoxy)ethoxy]ethyl}-1H-1,2,3-triazol-4-yl)methyl]-4-(pyren-1-yl)butanamide

This compound is prepared according to method A in 75% yield.

R_{f}=0.20 (EtOAc/MeOH 95/5)

¹H NMR (400 MHz, CDCl₃):

δ 8.22 (d, J=9.2 Hz, 1H, H-ar), 8.15-8.08 (m, 2H, H-ar), 8.04 (d, J=8.1 Hz, 2H, H-ar), 7.97 (s, 1H, H-triaz), 7.97-7.92 (m, 3H, H-ar), 7.79 (d, J=7.7 Hz, 1H, H-ar), 6.73 (bs, 1H, NH), 5.33 (dd, J=9.8 Hz, J=3.0 Hz, 1H, H-3), 5.26 (d, J=3.0 Hz, 1H, H-4), 5.12-5.04 (m, 2H, H-1, H-2), 4.51 (bs, 2H, CCH₂NH), 4.43 (bs, 2H, OCH₂CH₂N-triaz), 4.16 (q, J=6.4 Hz, 1H, H-5), 3.76 (bs, 2H, OCH₂CH₂N-triaz), 3.73-3.64 (m, 1H, ½ FucOCH₂CH₂O), 3.61-3.52 (m, 1H, ½ FucOCH₂CH₂O), 3.52-3.44 (m, 6H, FucOCH₂CH₂OCH₂CH₂O), 3.31 (t, J=6.6 Hz, 2H, PyrCH₂CH₂CH₂C(O)), 2.32, 2.17 (2 bs, 4H, PyrCH₂CH₂CH₂C(O)), 2.13, 2.00, 1.96 (3s, 3×3H, CH₃CO), 1.08 (d, J=6.4 Hz, 3H, CH₃).

¹³C NMR (100 MHz, CDCl₃):

δ 170.7, 170.5, 170.2 (3s, 3C, CH₃CO), 135.9 (C^{IV}-ar), 131.4 (C^{IV}-ar), 130.9 (C^{IV}-ar), 129.9 (C^{IV}-ar), 128.7 (C^{IV}-ar), 127.5 (CH-ar), 127.40 (s, 2C, CH-ar, CH-triaz), 127.38 (CH-ar) 126.7 (CH-ar), 125.9 (CH-ar), 125.1 (C^{IV}-ar), 125.0 (C^{IV}-ar), 124.9 (CH-ar), 124.83 (CH-ar), 124.79 (CH-ar), 123.4 (CH-ar), 96.2 (C-1), 71.2 (C-4) 70.55, 70.53, 70.2 (3s, 3C, FucOCH₂CH₂OCH₂CH₂O), 69.3 (OCH₂CH₂N-triaz), 68.2 (C-2), 68.0 (C-3), 67.3 (FucOCH₂CH₂O) 64.4 (C-5), 50.5 (OCH₂CH₂N-triaz), 36.1 (PyrCH₂CH₂CH₂C(O)), 35.1 (CCH₂NH), 32.8 (PyrCH₂CH₂CH₂C(O)), 27.5 (PyrCH₂CH₂CH₂C(O)), 20.9, 20.8, 20.7 (3s, 3C, CH₃CO), 15.9 (CH₃).

### (e) Preparation of Compound 5a (General Formula (I))

### N-[1-(2-{2-[2-(β-D-Galactopyranosyloxyethoxy)ethoxy]ethyl}-1H-1,2,3-triazol-4-yl)methyl]-4-(pyren-1-yl)butanamide

This compound is prepared according to method B in 70% yield.

¹H NMR (400 MHz, MeOD):

δ 8.23 (d, J=9.3 Hz, 1H, H-ar), 8.13 (d, J=3.0 Hz, 1H, H-ar), 8.11 (d, J=3.0 Hz, 1H, H-ar), 8.08-8.02 (m, 2H, H-ar), 7.97 (s, 1H, H-triaz), 7.94 (t, J=7.7 Hz, 3H, H-ar), 7.89 (bs, 1H, NH), 7.81 (d, J=7.7 Hz, 1H, H-ar), 4.49-4.44 (m, 4H, OCH₂CH₂N-triaz, CCH₂NH), 4.16 (d, J=7.5 Hz, 1H, H-1), 3.87-3.80 (m, 2H, H-4, ½ GalOCH₂CH₂O), 3.78-3.69 (m, 4H, H-6, OCH₂CH₂N-triaz), 3.56-3.48 (m, 2H, H-2, ½ GalOCH₂CH₂O), 3.48-3.41 (m, 2H, H-3, H-5), 3.40-3.34 (m, 6H, GalOCH₂CH₂OCH₂CH₂O), 3.31-3.27 (m, 2H, PyrCH₂CH₂CH₂C(O)), 2.38 (t, J=7.3 Hz, 2H, PyrCH₂CH₂CH₂C(O)), 2.19-2.06 (m, 2H, PyrCH₂CH₂CH₂C(O)).

¹³C NMR (100 MHz, MeOD):

δ 175.7 (C(O)NH), 137.3 (C^{IV}-ar), 132.7 (C^{IV}-ar), 132.2 (C^{IV}-ar), 131.2 (C^{IV}-ar), 129.8 (C^{IV}-ar), 128.51 (CH-ar), 128.48 (CH-ar), 128.4 (CH-ar), 127.6 (CH-ar), 127.0 (CH-ar), 126.1 (C^{IV}-ar), 126.0 (C^{IV}-ar), 125.9 (s, 2C, CH-ar), 125.8 (CH-ar), 124.4 (CH-ar), 105.0 (C-1), 76.6 (C-5), 74.8 (C-3), 72.4 (C-2), 71.21, 71.17, 71.1 (3s, 3C, GalOCH₂CH₂OCH₂CH₂O), 70.24 (C-4), 70.21 (OCH₂CH₂N-triaz), 69.5 (GalOCH₂CH₂O), 62.5 (C-6), 51.3 (OCH₂CH₂N-triaz), 36.6 (PyrCH₂CH₂CH₂C(O)), 35.6 (CCH₂NH), 33.7 (PyrCH₂CH₂CH₂C(O)), 29.0 (PyrCH₂CH₂CH₂C(O)).

### (f) Preparation of Compound 5b (General Formula (I))

### N-[1-(2-{2-[2-(β-D-Mannopyranosyloxyethoxy)ethoxy]ethyl}-1H-1,2,3-triazol-4-yl)methyl]-4-(pyren-1-yl)butanamide

This compound is prepared according to method B in 99% yield.

¹H NMR (400 MHz, DMSO-d₆+ε D₂O):

δ 8.35 (d, J=9.3 Hz, 1H, H-ar), 8.26 (dd, J=7.0 Hz, J=5.5 Hz, 2H, H-ar), 8.20 (dd, J=8.5 Hz, J=5.4 Hz, 2H, H-ar), 8.12 (d, J=2.0 Hz, 2H, H-ar), 8.05 (t, J=7.6 Hz, 1H, H-ar), 7.92 (d, J=7.8 Hz, 1H, H-ar), 7.87 (s, 1H, H-triaz), 4.60 (d, J=1.3 Hz, 1H, H-1), 4.46 (t, J=5.2 Hz, 2H, OCH₂CH₂N-triaz), 4.31 (s, 2H, CCH₂NH), 3.75 (t, J=5.2 Hz, 2H, OCH₂CH₂N-triaz), 3.66-3.26 (m, 16H, H-2, H-3, H-4, H-5, H-6, ManOCH₂CH₂OCH₂CH₂O, PyrCH₂CH₂CH₂C(O)), 2.28 (t, J=7.3 Hz, 2H, PyrCH₂CH₂CH₂C(O)), 2.06-1.95 (m, 2H, PyrCH₂CH₂CH₂C(O)).

¹³C NMR (100 MHz, DMSO-d₆+ε D₂O):

δ 172.3 (C(O)NH), 136.7 (C^{IV}-ar), 131.1 (C^{IV}-ar), 130.6 (C^{IV}-ar), 129.5 (C^{IV}-ar), 128.3 (C^{IV}-ar), 127.8 (CH-ar), 127.7 (CH-ar), 127.4 (CH-ar), 126.7 (CH-ar), 126.4 (CH-ar), 125.2 (2C, CH-ar), 125.0 (CH-ar), 124.4 (C^{IV}-ar), 124.3 (C^{IV}-ar), 123.7 (CH-ar), 123.3 (CH-triaz), 100.1 (C-1), 74.0, 70.9, 70.3 (C-5, C-2, C-3), 69.8, 69.7, 69.6 (ManOCH₂CH₂OCH₂CH₂O), 69.0 (OCH₂CH₂N-triaz), 67.0 (C-4), 65.8 (GalOCH₂CH₂O), 61.3 (C-6), 49.5 (OCH₂CH₂N-triaz), 35.1 (PyrCH₂CH₂CH₂C(O)), 34.2 (CCH₂NH), 32.4 (PyrCH₂CH₂CH₂C(O)), 27.8 (PyrCH₂CH₂CH₂C(O)).

### (g) Preparation of Compound 5c (General Formula (I))

### N-[1-(2-{2-[2-(α-L-Fucopyranosyloxyethoxy)ethoxy]ethyl}-1H-1,2,3-triazol-4-yl)methyl]-4-(pyren-1-yl)butanamide

This compound is prepared according to method B in 99% yield.

¹H NMR 400 MHz DMSO-d₆+ε D₂O):

δ 8.35 (d, J=9.3 Hz, 1H, H-ar), 8.30-8.24 (m, 2H, H-ar), 8.22 (d, J=4.2 Hz, 1H, H-ar), 8.20 (d, J=5.8 Hz, 1H, H-ar), 8.12 (d, J=2.0 Hz, 2H, H-ar), 8.05 (t, J=7.8 Hz, 1H, H-ar), 7.93 (d, J=7.8 Hz, 1H, H-ar), 7.88 (s, 1H, H-triaz), 4.59 (d, J=2.7 Hz, 1H, H-1), 4.46 (t, J=5.2 Hz, 2H, OCH₂CH₂N-triaz), 4.32 (s, 2H, CCH₂NH), 3.76 (t, J=5.2 Hz, 3H, OCH₂CH₂N-triaz, H-5), 3.59-3.37 (m, 14H, H-2, H-3, H-4, H-6, ManOCH₂CH₂OCH₂CH₂O), 3.33-3.26 (m, 2H, PyrCH₂CH₂CH₂C(O)), 2.28 (t, J=7.3 Hz, 2H, PyrCH₂CH₂CH₂C(O)), 2.06-1.96 (m, 2H, PyrCH₂CH₂CH₂C(O)), 1.03 (d, J=6.5 Hz, 3H, CH₃).

¹³C NMR (100 MHz, DMSO-d₆+ε D₂O):

δ 172.1 (C(O)NH), 136.7 (C^{IV}-ar), 131.0 (C^{IV}-ar), 130.6 (C^{IV}-ar), 129.4 (C^{IV}-ar), 128.3 (C^{IV}-ar), 127.7 (CH-ar), 127.6 (CH-ar), 127.4 (CH-ar), 126.7 (CH-ar), 126.3 (CH-ar), 125.1 (2C, CH-ar), 124.9 (CH-ar), 124.4 (C^{IV}-ar), 124.3 (C^{IV}-ar), 123.7 (CH-ar), 123.3 (CH-triaz), 99.4 (C-1), 71.6 (C-4), 69.8, 69.6 (2s, 3C, FucOCH₂CH₂OCH₂CH₂O), 69.58 (C-2 or C-3), 68.9 (OCH₂CH₂N-triaz), 68.0 (C-2 or C-3), 66.7 (GalOCH₂CH₂O), 66.0 (C-5), 49.5 (OCH₂CH₂N-triaz), 35.0 (PyrCH₂CH₂CH₂C(O)), 34.2 (CCH₂NH), 32.4 (PyrCH₂CH₂CH₂C(O)), 27.7 (PyrCH₂CH₂CH₂C(O)), 16.6 (CH₃).

### EXAMPLE II

### Fabrication of Electronic Nano-Detection Devices and their Use for the Detection of Lectins

1) Fabrication of Electronic Nano-Detection Devices Respectively Named “SWNT-FET” and “CCG-FET”.

The used carbon nanostructures are respectively the carbon nanotubes (more particularly single-walled carbon nanotubes (SWNTs)) and the graphene.

Single-walled carbon nanotubes (SWNTs) were procured from Carbon Solutions Inc. and were used as conducting channels in the field-effect transistor (FET) devices (FETs) as described below.

Chemically reduced graphene oxide, which is also known in the literature as chemically converted graphene (CCG), was prepared as previously described in the literature⁴⁻⁶. Briefly, graphite oxide was synthesized utilizing a modified Hummers' method on graphite flakes (Sigma Aldrich) that underwent a preoxidation step.⁵ Graphite oxide (˜0.125 wt %) was exfoliated to form graphene oxide via 30 minutes of ultrasonification followed by 30 minutes of centrifugation at 3400 revolutions per minute (r.p.m.) to remove unexfoliated graphite oxide (GO). Graphene oxide was then reduced to RGO with hydrazine hydrate (Sigma Aldrich) following the reported procedure^{4,6}, the chemically converted graphene (CCG) thus obtained being then used as conducting channels in the FETs.

Metal interdigitated devices (Au/Ti, 100 nm/30 nm) with interelectrode spacing of 10 μm were patterned on a Si/SiO₂ substrate using conventional photolithography (FIGS. 3(c) and 3(d)). Each chip (2 mm×2 mm) containing four identical devices was then set into a 40-pin ceramic dual in-line package (CERDIP) and wire-bonded using Au wire. Devices were subsequently isolated from the rest of the package by epoxying the inner cavity.

SWNTs were deposited onto each interdigitated microelectrodes pattern by a.c. dielectrophoresis (DEP) method from a suspension in N,N-dimethylformamide (DMF) (FIG. 3(b)) (Agilent 33250A 80 MHz Function/Arbitrary Waveform Generator, a.c. frequency (10 MHz), bias voltage (8 Vₚₚ), bias duration (60 s)).⁷

CCG devices were prepared using the same DEP technique (FIG. 3(b)) but with different parameters (a.c. frequency (300 kHz), bias voltage (10.00 Vₚₚ), bias duration (120s)).⁸

The electrical performance of each such obtained “SWNT-FET” device or “CCG-FET” device was investigated in electrolyte gated FET device configuration. The conductance of each FET device was tuned using electrolyte as a highly effective gate.

Two Keithley 2400 sourcemeters were used for FET measurements.

A small fluid chamber (1 mL) was placed over the “SWNT-FET” device or the “CCG-FET” device to control the liquid environment using phosphate buffer solution (PBS) at pH 7. A liquid gate potential (−0.75 V to +0.75 V) with respect to the grounded drain electrode was applied using an Ag/AgCl (3 M KCl) reference electrode submerged in the gate electrolyte.

The drain current of the device was measured at a constant source-drain voltage (50 mV).

Transfer characteristics (conductance (G) versus gate voltage (V_{g})) were measured to investigate the interactions between pyrene-based glycoconjugates functionalized carbon nanomaterials and lectins (FIGS. 4 and 6).

2) Non Covalent Functionalization of SWNT-FET or CCG-FET with Pyrene Glycoconjugates (1)

To selectively detect lectins, the surface of the SWNT-FET device or the CCG-FET device thus obtained is non covalently functionalized with respectively the three pyrene-based glycoconjugates (I) (5a to 5c) such as prepared in example I.

The [symbol] (or carbohydrate) which is present at the extremity of each of these glycoconjugates (I) is respectively the β-D-galactosyl (for glycoconjugate 5a), the α-D-mannosyl (for 5b) and the α-L-fucosyl (for 5c).

Here is thus investigated the specific interactions between three different sugars, namely β-D-galactose, α-D-mannose and α-L-fucose with respectively the three following lectins: PA-IL, ConA, and PA-IIL, by using the above mentioned non covalently functionalized SWNT-FET device or CCG-FET device (see FIG. 3(a)).

PA-IL is a bacterial lectin isolated from Pseudomonas aeruginosa that is specific for β-D-galactose and expressed in recombinant form in Escherichia coli.

PA-IIL is a bacterial lectin isolated from Pseudomonas aeruginosa that is specific for α-L-fucose and expressed in recombinant form in Escherichia coli.

These lectins PA-IL and PA-IIL were produced by the Inventors according to previously reported procedures⁹.

ConA (25 kDa) is a plant lectin from Canavalia ensiformis that is specific for α-D-mannose and is available commercially: it was purchased from Sigma and used without further purification.

Surface functionalization of SWNT-FET device or CCG-FET device with each pyrene based glycoconjugate (5a to 5c) was performed by incubating the chips in 20 μM of the pyrene glycoconjugates solution (in deionized water) for 2 hr followed by rinsing three times with double-distilled water. After testing the transfer characteristics, the chips were incubated for 40 min in different concentrations of lectin solutions prepared in PBS with 5 μM CaCl₂ and subsequently washed three times with PBS solution. For each glycoconjugate functionalized device, non-specific lectins were tested first, followed by washing procedures and measuring of specific lectin. The final transfer characteristics were tested again in the configuration mentioned above.

Imaging studies: The scanning electron microscopy (SEM) was performed with a Phillips XL30 FEG at acceleration voltage of 10 keV (FIG. 3(d)).

Atomic force microscope (AFM) images (FIGS. 5 and 7) were obtained using scanning probe microscope (Veeco Nanoscope II) in a tapping mode configuration. Samples were prepared by spin-coating bare SWNTs or CCGs onto a poly-L-lysine treated freshly cleaved sheet of mica substrate. The bare SWNTs and CCGs images were taken after 45 min of drying in ambient. Glycoconjugates functionalization was performed by incubating the SWNTs or RGO deposited mica substrate with 20 μM glycoconjugate in deionized water solution for 2 hr at room temperature. Images of functionalized SWNTs and RGO were taken after washing the substrate with DI water and drying in ambient for 45 min. Interaction with specific lectin was investigated by incubating the treated substrate with 2 μM lectin solution (in PBS with 5 μM CaCl₂) and subsequent washing with PBS solution and drying in ambient for 45 min.

3) Results and Discussion

The electronic detection of the interactions between the sugar (carbohydrate) of the glycoconjugates (I) and lectin molecules is illustrated by the curves of the FIGS. 4 and 6.

FIGS. 4 and 6 show the conductance G vs V_{g} curves for respectively CCG-FET and SWNT-FET at different stages of glycoconjugate lectin interactions.

Upon interaction with pyrene-based glycoconjugates (5a to 5c), a decrease in the CCG-FET device conductance with a slight negative shift in gate voltage was observed (FIG. 4). The decrease in device conductance can be attributed to the electron donation from pyrene molecules to CCG conducting channel.

The response of the CCG-FET devices after glycoconjugate functionalization was selective to lectins. For example, FIG. 4(b) shows the response of β-D-galactose pyrene-based glycoconjugate (5a) devices to two lectins. Upon incubation with non-specific lectin (ConA) the transfer characteristics remained unaffected. However, when treated with the mannose specific lectin (PA-IL) a decrease in conductance was observed indicating the selective interaction between the glycoconjugate and the lectin. Similar results were observed with α-D-mannose and α-L-fucose pyrene-based glycoconjugates (FIG. 4(a) and FIG. 4(c)).

Similar experiments were performed with SWNT-FET devices. As presented in FIG. 6, a decrease in device conductance can be observed upon interaction with pyrene-glycoconjugates (5a and 5b). Upon treatment with non-specific lectins, the transfer characteristics of the SWNT-FET devices remained unaffected. A decrease in device conductance was observed after treatment with specific lectin, indicating selective interaction between lectins and glycoconjugates.

Additionally, the sensitivity of CCG-FET devices was investigated by plotting the G vs Vg for β-D-galactose glycoconjugate (5a) functionalized device (control measurements with 10 μM ConA) for varying concentration (2 nM to 10 μM) of specific lectin PA-IL (FIG. 4(d)). The CCG-FET device response to 10 μM specific lectin PA-IL is almost two times higher than the response to 10 μM non-specific lectin ConA, further demonstrating good selectivity.

Atomic force microscopy (AFM) imaging was performed to study the surface morphology of the CCG at different stages of functionalization. Bare CCG was observed to be 0.67±0.15 nm in thickness (FIG. 5(a)). After functionalization with α-D-mannose glycoconjugates (5b), the total height increased to 2.44±0.35 nm (FIG. 5(b)). Later, after exposing the glycoconjugate functionalized CCG to specific binding lectin (ConA for α-D-mannose), an increase in height to 8.25±1.73 nm was observed (FIG. 5(c)). Typically, ConA is observed as a tetramer in solution at pH ≧7 and the molecular dimensions of tetramer are 60×70×70 A (Protein DataBank, 1CN1) from X-ray diffraction studies. The height measurements obtained by AFM are in good agreement with the literature values.

Additionally, AFM imaging was performed to investigate the surface morphology of the SWNTs at different stages of functionalization. The height SWNTs was observed to be around 3-4 nm indicating the presence of SWNTs bundles (FIG. 7(a)). After functionalization with α-D-mannose glycoconjugates (5b), the total height increased to 5-7 nm (FIG. 7(b)). Later, after exposing the glycoconjugate functionalized SWNTs to specific binding lectin (ConA for α-D-mannose), an increase in height of more than 10 nm was observed (FIG. 7(c)), indicating adsorption of lectins onto the SWNTs network.

In conclusion, we have demonstrated the electronic detection of interactions between pyrene-based glycoconjugates and bacterial lectins using CCG-FET and SWNT-FET devices. The interaction between lectins and glycoconjugates was transduced as conductance change in CCG-FET and SWNT-FET devices.

### REFERENCES

(1) Szurmai, Z.; Szabó, L.; Lipták, A. Acta Chim. Hung. 1989, 126, 259-269.
(2) Li, J.; Zacharek, S.; Chen, X.; Wang, J.; Zhang, W.; Janczuk, A.; Wang, P. G. Bioorg. Med. Chem. 1999, 7, 1549-1558.
(3) Sanki, A. K.; Mahal, L. K. Synlett 2006, 455-459.
(4) Li, D. et al. Nature Nano 2008, 3, 101-105.
(5) Kovtyukhova, N. I. et al. Chem. Mater. 1999, 11, 771-778.
(6) Kotchey, G. P. et al. Enzymatic oxidation of graphene oxide. ACS Nano 2011, 5, 2098-2108.
(7) Vedala H. et al. Nano Lett. 2011, 11, 170-175.
(8) Vijayaraghavan, A. et al. ACS Nano 2009, 3, 1729-1734.
(9) (a) Blanchard, B. et al. J. Mol. Biol. 2008, 383, 837-853. (b) Mitchell, E. P. et al. Proteins: Struct. Funct. Bioinfo. 2005, 58, 735-746.

1. Non covalent molecular structure characterized in that it comprises a carbon nanostructure and a pyrene based glycoconjugate (I) which is linked to the said carbon nanostructure by a non covalent link,
the said glycoconjugate (I) having the formula: 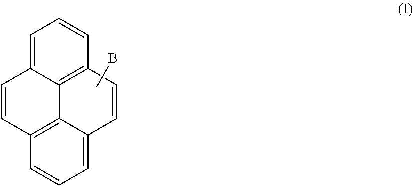
wherein
B is a group which is present on any of the ten carbon atoms of the pyrene structure represented in (I) susceptible to bear a substituent, and is represented by the following group:
—(CH₂)ₙ—CO—NH-A,
wherein
n is an integer from 1 to 9,
A is a group of formula: 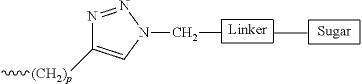
wherein
p is an integer from 1 to 9,
the [symbol] is a group of formula: 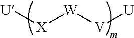
wherein
m is an integer from 0 to 15,
U′, U=absent or is CH₂ with the proviso that when m=0 then if one of U′
or U is absent then the other is CH₂,
X=CH₂, O, CO (carbonyl),
W=CH₂, NH,
V=CH₂, C₆H₄ (phenyl “Ph”),
the [symbol] is a group having at least one carbohydrate moiety and is selecting in
the group comprising: 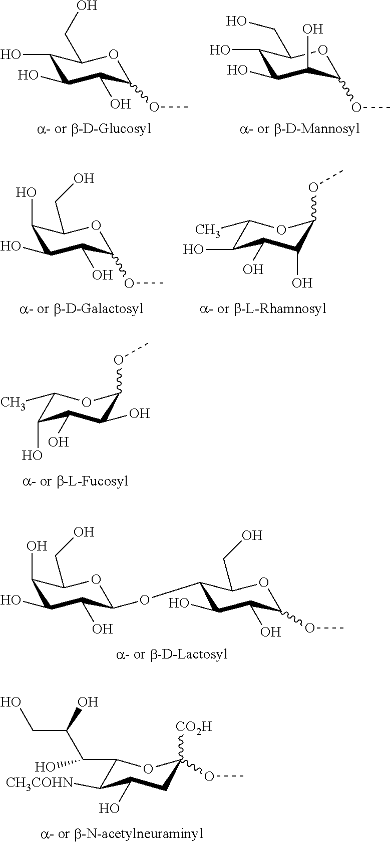
and their derivatives.

2. Non covalent molecular structure according to claim 1, wherein the sugar derivatives in the A group are selected in the group comprising:

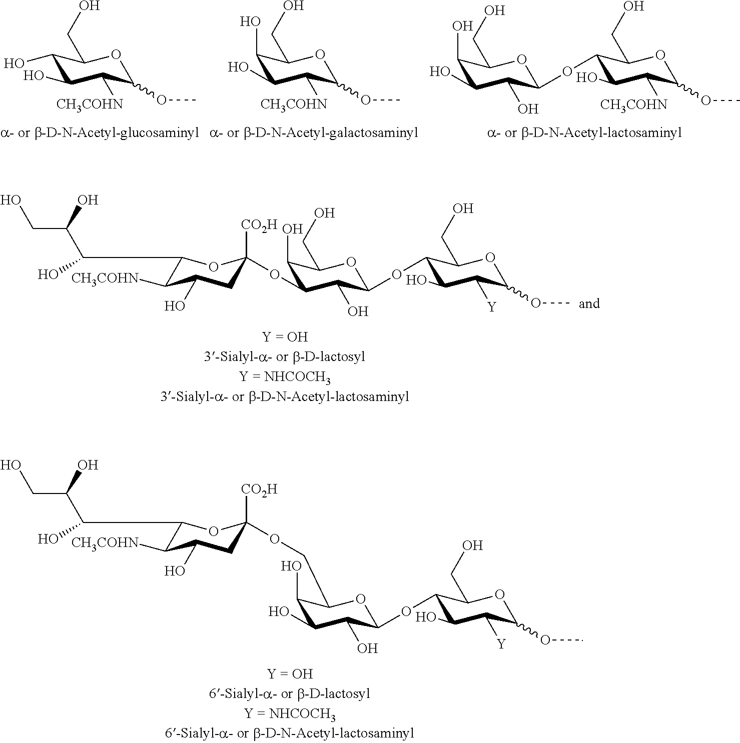

3. Non covalent molecular structure according to claim 1, wherein the sugar derivatives in the A group are selected in the group comprising:

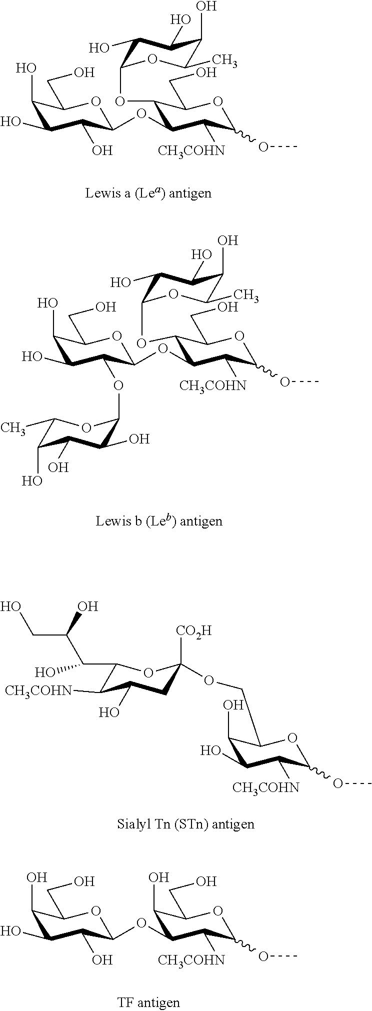

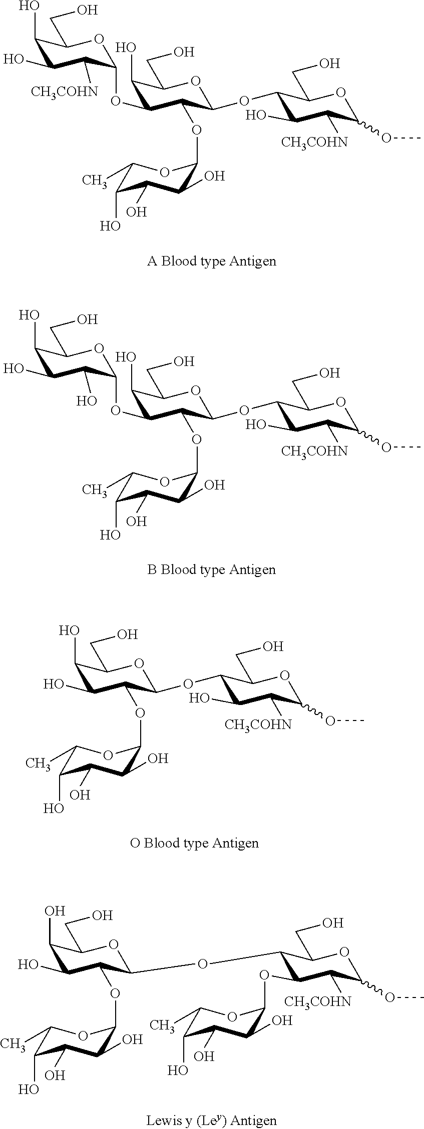

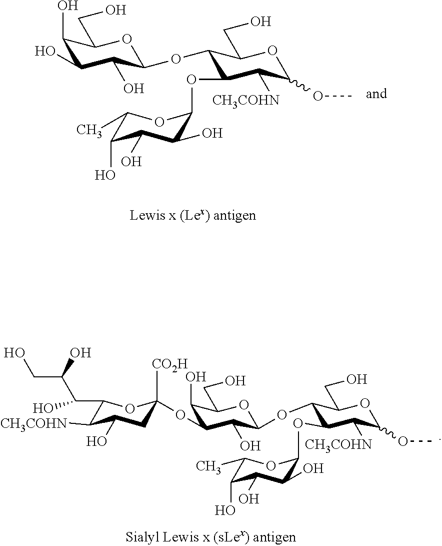

4. Non covalent molecular structure according to claim 1, wherein the [symbol] defined in the A group is selected in the group comprising:
m=0, U′=absent and U=CH₂,
m=0, U′=U=CH₂,
m=1, U′=U=absent, X=W=V=CH₂,
m=2, U′=U=absent, X=W=V=CH₂,
m=1, U′=CH₂, U=absent, X=O, W=V=CH₂,
m=2, U′=CH₂, U=absent, X=O, W=V=CH₂,
m=2, U′=absent, U=V=CH₂, X=CO, W=NH and
m=1, U′=U=absent, X=CO, W=NH and V=Ph.

5. Non covalent molecular structure according to claim 1, wherein in the pyrene based glycoconjugate (I), the integer n is 3, the integer p is 1 and the said glycoconjugate (I) is represented by the formula:

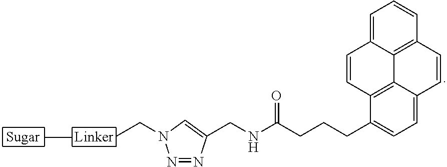

6. Non covalent molecular structure according to claim 5, wherein in the pyrene based glycoconjugate (I):
the [symbol] is CH₂—(O—CH₂—CH₂)₂ (m=2, U′=CH₂, U=absent, X=O, W=V=CH₂),
the sugar is selected in the group comprising β-D-galactosyl, α-D-mannosyl and α-L-fucosyl.

7. Non covalent molecular structure according to claim 1, wherein the carbon nanostructures are selected in the group comprising carbon nanotubes, graphene, graphitic onions, cones, nanohorns, nanohelices, nanobarrels and fullerenes.

8. Non covalent molecular structure according to claim 7, wherein the carbon nanostructures are graphene and carbon nanotubes, the said carbon nanotubes being selected in the group comprising Single Wall Carbon Nanotubes (SWCNTs), Double Wall Carbon Nanotubes (DWCNTs), Triple Wall Carbon Nanotubes (TWCNTs) and Multi Wall Carbon Nanotubes (MWCNTs).

9. A device for detecting a lectin characterized in that it comprises a non covalent molecular structure according to claim 1.

10. A device according to claim 9 which is an electronic nano-detection device and which comprises a field effect transistor (FET),
the said device comprising:
carbon nanostructures bridging two metal electrodes respectively called “source” (S) and “drain” (D),
a third electrode called “gate” (G) connected either to a substrate layer or to an electrode immersed in a solution covering the said device (“liquid gate”).

11. A device according to claim 10 wherein the two metal electrodes (S) and (D) are spacing each other from 1 nm to 10 cm, preferably from 1 cm to 2.5 cm and more preferably from 1 μm to 10 μm.

12. A device according to claim 10, wherein the substrate layer is an insulator.

13. Method for detecting the presence of a lectin in a sample to be analysed characterized in that it comprises the following steps:
using a device according to claim 9,
bringing the lectin to be analysed in contact with the non covalent molecular structure according,
detecting a molecular interaction between the lectin and the sugar of the pyrene based glycoconjugate (I) of the said non covalent molecular structure, said molecular interaction being detected by a change of the conductive properties of the carbon nanostructures resulting in a change of the electric signal of the said device.

14. Method according to claim 13, wherein the lectin is selected in the group comprising Pseudomonas aeruginosa first lectin (PA-IL), Pseudomonas aeruginosa second lectin (PA-IIL), Concanavalin A (Con A) lectin, Burkholderia cenocepacia A (Bc2L-A) lectin, Burkholderia cenocepacia B (Bc2L-B) lectin, Burkholderia cenocepacia C (Bc2L-C) lectin, Burkholderia ambifaria (Bamb541) lectin, Ralstonia solanacearum (RSL) lectin, Ralstonia solanacearum second lectin (RS-IIL) and Chromobacterium violaceum (CV-IIL) lectin.

15. Method according to anyone of claim 13, wherein the preparation of the device as defined in anyone of claims 10 to 12 comprises the following steps:
forming two metal electrodes (S) and (D) on the substrate layer connected to (G),
adding, between the two electrodes (S) and (D), the carbon nanostructures and then a pyrene based glycoconjugate (I) in order to form a non covalent molecular structure.

16. Method according to claim 13, wherein the preparation of the device as defined in anyone of claims 10 to 12 comprises the following steps:
forming two metal electrodes (S) and (D) on the substrate layer connected to (G),
adding, between the two electrodes (S) and (D), a non covalent molecular structure.

17. Method according to claim 13, wherein the preparation of the device comprises the following steps:
generating carbon nanostructures on the substrate layer connected to (G) (by a chemical vapour deposition (CVD) process),
forming two metal electrodes (S) and (D) around the carbon nanostructures,
adding a pyrene based glycoconjugate (I) in order to form a non covalent molecular structure.
